(19) United States
(12) Patent Application Publication
(10) Pub. No.: US 2013/0217594 A1
(43) Pub. Date: Aug. 22, 2013

(54) METHODS FOR SCREENING COMPOUNDS FOR CAPABILITY TO INHIBIT PREMALIGNANT SQUAMOUS EPITHELIAL CELL PROGRESSION TO A MALIGNANT STATE

(71) Applicants: Margie Clapper, Harleysville, PA (US); Ekaterina Shatalova, Philadelphia, PA (US)

(72) Inventors: Margie Clapper, Harleysville, PA (US); Ekaterina Shatalova, Philadelphia, PA (US)

(73) Assignee: Institute for Cancer Research d/b/a The Research Center of Fox Chase Cancer (Fox Chase Cancer, Philadelphia, PA (US)

(21) Appl. No.: 13/882,299

(22) PCT Filed: Oct. 28, 2011
(86) PCT No.: PCT/US2011/058306

§ 371 Date: Apr. 29, 2013

Related U.S. Application Data
(60) Provisional application No. 61/408,019, filed on Oct. 29, 2010.

(51) Int. Cl. C12Q 1/26

(52) U.S. Cl. CPC C12Q 1/26
USPC 506/10; 435/6.12; 435/25; 435/7.1; 435/7.9

(57) ABSTRACT

Methods for screening compounds for their capability to inhibit CYP1B1-mediated proliferation and motility of and/or to reverse estrogen-induced reduction in apoptosis of premalignant or malignant cells are provided. Kits for practicing the screening methods are also provided.

### CROSS REFERENCE TO RELATED APPLICATIONS

This application claims priority to U.S. Provisional Application No. 61/408,019 filed on Oct. 29, 2010, the entire contents of which are incorporated by reference herein, in their entirety and for all purposes.

### STATEMENT OF GOVERNMENT SUPPORT

The inventions described herein were made, in part, with funds obtained from the National Cancer Institute, Grant Nos. CA-006927 and CA-113451. The U.S. government may have certain rights in these inventions.

### FIELD OF THE INVENTION

The invention relates generally to the field of early stage cancer drug research. More particularly, the invention relates to methods for identifying compounds that are capable of inhibiting the progression of premalignant cells to a malignant state, and kits for practicing these methods.

### BACKGROUND OF THE INVENTION

Various publications, including patents, published applications, technical articles and scholarly articles are cited throughout the specification. Each of these cited publications is incorporated by reference herein, in its entirety and for all purposes.

Head and neck cancer, currently the sixth most common cancer in the U.S., accounts for 650,000 new cancer cases each year worldwide. Head and neck cancer is a heterogeneous group of malignancies that develop primarily in the squamous epithelium of the lip, oral cavity, pharynx, larynx, nasal cavity, and paranasal sinuses. A rise in the incidence of squamous cell carcinoma (SCC) of the head and neck (HNSCC) in adults age 40 or less has been reported and attributed primarily to an increase in the prevalence of tongue cancers.

Exposure to tobacco smoke and use of alcohol are among the major risk factors for developing HNSCC. Infection with human papilloma virus (HPV) has been associated with a subset of HNSCCs, SCC of the oropharynx. The lack of an association of a substantial proportion of HNSCC cases with exposure to these established risk factors, however, suggests that additional genetic and/or environmental factors may contribute to disease susceptibility. Recent data suggests that 75% of young, non-smoker/non-drinker HNSCC patients who develop oral tongue SCC (not associated with HPV) are female. Thus, in addition to the major risk factors, female hormones may contribute to head and neck carcinogenesis.

CYP1B1 is an enzyme that, along with CYP1A1 and CYP3A4, catalyzes the formation of carcinogenic metabolites of 17β-estradiol (E2) and of constituents of tobacco smoke that are subsequently inactivated by one or more detoxification enzymes, including catechol-o-methyltransferase (COMT), sulfotransferase (SULT)1A1, UDP-glucuronosyltransferase (UGT)1A1 and glutathione-S-transferase (GST)P1. Little attention has been given to the importance of the estrogen metabolism pathway in HNSCC.

The role CYP1B1 may play in the invasive potential of endometrial cancer cells was investigated, with some evidence that CYP1B1 affects the cellular proliferation of endometrial cancer cells and that CYP1B1 influences the invasive properties of endometrial carcinomas (Saini S et al. (2009) Cancer Res. 69:7038-45). It is believed that the role of CYP1B1 in motility has not been previously investigated. Invasive potential and motility are distinguishable aspects of cancer development and progression (Kassis J et al. (2001) Cancer Biology 11:105-17).

There is a need to identify agents for preventive intervention in the cellular progression from a precancerous state to a cancerous state, and a need for suitable models for testing agents for efficacy in inhibiting or preventing the cell's progression from a precancerous state to a cancerous state.

### SUMMARY OF THE INVENTION

The invention provides methods for screening compounds for the ability to inhibit progression of a premalignant cell to a malignant state, to inhibit motility and proliferation, and/or to induce apoptosis, by affecting one or more of CYP1B1 gene transcription, CYP1B1 protein expression, CYP1B1 biologic activity, or estrogen-inhibited apoptosis.

In one embodiment, the methods comprise contacting premalignant cells or malignant cells exposed to an amount of an estrogen effective to inhibit apoptosis, examples of such as an estrogen being an estrogen hormone, a phytoestrogen, a mycoestrogen, a xenoestrogen, or a combination thereof, with a test compound, and measuring the level of apoptosis in the cells in the presence of the test compound relative to the level of apoptosis in the cells in the absence of the test compound, wherein an increase in apoptosis in the presence of the test compound indicates that the test compound is capable of restoring apoptosis inhibited by an estrogen in the premalignant cells, and wherein a decrease in apoptosis in the presence of the test compound indicates that the test compound is capable of further inhibiting apoptosis in the premalignant cells or in the malignant cells.

In some embodiments, the methods comprise contacting premalignant cells expressing CYP1B1 or malignant cells expressing CYP1B1 with a test compound, and measuring the level of motility of the cells in the presence of the test compound relative to the level of motility of the cells in the absence of the test compound, wherein a decrease in motility in the presence of the test compound indicates that the test compound is capable of inhibiting motility of the premalignant cells or of the malignant cells, and wherein an increase in motility in the presence of the test compound indicates that the test compound is capable of enhancing motility of the premalignant cells or of the malignant cells.

In some embodiments, the methods comprise contacting premalignant cells expressing CYP1B1 or malignant cells expressing CYP1B1 with a test compound, and measuring the level of proliferation of the cells in the presence of the test compound relative to the level of proliferation of the cells in the absence of the test compound, wherein a decrease in proliferation in the presence of the test compound indicates that the test compound is capable of inhibiting proliferation of the premalignant cells or of the malignant cells, and wherein an increase in proliferation in the presence of the test compound indicates that the test compound is capable of enhancing proliferation of the premalignant cells or of the malignant cells.

In some embodiments, the methods comprise contacting premalignant cells having the CYP1B1 gene or malignant cells having the CYP1B1 gene with a test compound and an agent capable of upregulating CYP1B1 transcription, and measuring the level of CYP1B1 mRNA in the cells in the presence of the test compound relative to the level of CYP1B1 mRNA in the cells in the absence of the test compound, wherein a decrease in the level of CYP1B1 mRNA in the presence of the test compound indicates that the test compound is capable of inhibiting CYP1B1 transcription in the premalignant cells or in the malignant cells, and wherein an increase in the level of CYP1B1 mRNA in the presence of the test compound indicates that the test compound is capable of enhancing CYP1B1 transcription in the premalignant cells or in the malignant cells.

In some embodiments, the methods comprise contacting premalignant cells having the CYP1B1 gene or malignant cells having the CYP1B1 gene with a test compound and an agent capable of upregulating CYP1B1 protein expression, and measuring the level of CYP1B1 protein in the cells in the presence of the test compound relative to the level of CYP1B1 protein in the cells in the absence of the test compound, wherein a decrease in the level of CYP1B1 protein in the presence of the test compound indicates that the test compound is capable of inhibiting CYP1B1 protein expression in the premalignant cells or in the malignant cells, and wherein an increase in the level of CYP1B1 protein in the presence of the test compound indicates that the test compound is capable of enhancing CYP1B1 protein expression in the premalignant cells or in the malignant cells.

In some embodiments, the methods comprise contacting premalignant cells having the CYP1B1 gene or malignant cells having the CYP1B1 gene with a test compound and an agent capable of upregulating CYP1B1 biologic activity, and measuring the level of CYP1B1 biologic activity in the cells in the presence of the test compound relative to the level of CYP1B1 biologic activity in the cells in the absence of the test compound, wherein a decrease in the level of CYP1B1 biologic activity in the presence of the test compound indicates that the test compound is capable of inhibiting CYP1B1 biologic activity in the premalignant cells or in the malignant cells, and wherein an increase in the level of CYP1B1 biologic activity in the presence of the test compound indicates that the test compound is capable of enhancing CYP1B1 biologic activity in the premalignant cells or in the malignant cells.

Any of these methods may be carried out using premalignant cells alone, or malignant cells alone. Premalignant cells are preferred. The premalignant cells may be premalignant epithelial cells, and may be capable of progressing to a malignancy of the head and neck such as a squamous cell carcinoma of the head and neck. The premalignant cells may be premalignant squamous epithelial cells of the lip, oral cavity, pharynx, larynx, nasal cavity, or paranasal sinuses. The malignant cells may be any cancer cell, and are preferably squamous cell carcinoma of the head and neck cells. The malignant cells may be malignant squamous epithelial cells of the lip, oral cavity, pharynx, larynx, nasal cavity, or paranasal sinuses.

The invention also provides kits for practicing methods for screening compounds. In one embodiment, a kit comprises a premalignant and/or a malignant squamous epithelial cell of the lip, oral cavity, pharynx, larynx, nasal cavity, or paranasal sinuses, an estrogen such as an estrogen hormone, a phytoestrogen, a mycoestrogen, or a xenoestrogen, and instructions for using the kit in a method for screening test compounds for capability of restoring apoptosis inhibited by an estrogen in a premalignant or in a malignant squamous epithelial cell of the lip, oral cavity, pharynx, larynx, nasal cavity, or paranasal sinuses.

In some embodiments, a kit comprises a premalignant and/or a malignant squamous epithelial cell of the lip, oral cavity, pharynx, larynx, nasal cavity, or paranasal sinuses, optionally, an activator of CYP1B1 biologic activity, and instructions for using the kit in a method for screening test compounds for capability of modulating motility of a premalignant or a malignant squamous epithelial cell of the lip, oral cavity, pharynx, larynx, nasal cavity, or paranasal sinuses and/or instructions for using the kit in a method for screening test compounds for capability of modulating proliferation of a premalignant or a malignant squamous epithelial cell of the lip, oral cavity, pharynx, larynx, nasal cavity, or paranasal sinuses.

In some embodiments, a kit comprises a premalignant and/or a malignant squamous epithelial cell of the lip, oral cavity, pharynx, larynx, nasal cavity, or paranasal sinuses having the CYP1B1 gene, an agent capable of upregulating CYP1B1 transcription and/or an agent capable of upregulating CYP1B1 protein expression and/or an agent capable of upregulating CYP1B1 biologic activity, and instructions for using the kit in a method for screening test compounds for capability of modulating CYP1B1 transcription, protein level, or biologic activity in a premalignant or a malignant squamous epithelial cell of the lip, oral cavity, pharynx, larynx, nasal cavity, or paranasal sinuses. The cell having the CYP1B1 gene may be a stably or transiently transformed cell or a cell line.

### BRIEF DESCRIPTION OF THE DRAWINGS

FIG. 1A shows representative images of human premalignant (MSK-Leuk1) and malignant (SCC) head and neck cells stained with antibodies against CYP1B1, ERα and ERβ. Secondary antibody alone was used as a negative control (not shown); Magnification 40×. FIG. 1B shows the detection of ERα, ERβ and CYP1B1 in MSK-Leuk1, HNSCC and MCF-7 cells by Western blot. FIG. 1C shows the expression of estrogen metabolism genes in cultured human premalignant and malignant head and neck cells. Values (2^{-ΔCt}) represent transcript levels (±standard deviation), normalized to the internal control (TFRC).

FIG. 2A shows the effect of E2 (1 nM for 24 hrs) on the expression of ERβ, CYP1B1 and COMT in cultured human premalignant (MSK-Leuk1) and malignant (SCC) head and neck cells. FIG. 2B shows a time course of the effect of E2 treatment (1 nM) on CYP1B1 transcript levels in MSK-Leuk1 cells. Cells were incubated in phenol red free (MSK-Leuk1 and HNSCC cells) and charcoal-stripped serum supplemented media (HNSCC cells) for 3 days prior to E2 exposure. Bars represent mean percent (±standard error) relative to vehicle-treated control (100%).

FIG. 3 shows that CYP1B1 deficiency decreases the motility of MSK-Leuk1 cells. FIG. 3A shows the detection of CYP1B1 in vector-expressing and CYP1B1 shRNA-expressing cells by Western blot. Stable clones were selected with puromycin for 1 week, and expanded and analyzed using antibodies specific for CYP1B1. FIG. 3B shows representative images of cell monolayers at baseline (0 hours) and 16 hours post-scratch, treated with vehicle (0.01% ethanol) or E2 (1 nM). A similar response was observed for vehicle- and E2-treated cells. FIG. 3C shows a percentage of gap closure calculated as (area at 16 h−area at 0 h)/(area at 0 h) in CYP1B1 shRNA-expressing cells and vector-expressing cells treated with vehicle or E2. Gap area was calculated as a mean of 3 replicates. FIG. 3D shows apoptosis in CYP1B1 shRNA-expressing cells and vector-expressing cells during the 16-h period, as measured using a Nexin kit (Millipore). FIG. 3E shows proliferation of CYP1B1 shRNA-expressing cells and vector-expressing cells during the 16-h period, measured using a Fluorescent DNA Quantitation kit (BioRad). All bars represent the mean of 3 replicates ±standard error.

FIG. 4 shows the effect of E2 and CYP1B1 on the proliferation and apoptosis of MSK-Leuk1 cells. Cells were incubated in phenol red-free and serum-free medium containing either 1 nM E2 or vehicle (0.01% ethanol) for 72 h. FIG. 4A shows that CYP1B1 deficiency inhibits proliferation of MSK-Leuk1 cells (total DNA). FIG. 4B shows that exposure to E2 inhibits apoptosis of MSK-Leuk1 cells (annexin). FIG. 4C shows that Fulvestrant (1 μM) restores E2-mediated decrease of apoptosis in MSK-Leuk1 cells. All bars represent the mean of 3 replicates ±standard error.

FIG. 5 shows representative images of human head and neck tissues from TMAs stained with antibodies against CYP1B1, ERα, ERβ and E2. Secondary antibody alone was used as a negative control (not shown); Magnification 20×.

### DETAILED DESCRIPTION OF THE INVENTION

Various terms relating to aspects of the present invention are used throughout the specification and claims. Such terms are to be given their ordinary meaning in the art, unless otherwise indicated. Other specifically defined terms are to be construed in a manner consistent with the definition provided herein.

As used herein, the singular forms “a,” “an,” and “the” include plural referents unless expressly stated otherwise.

Premalignant or precancerous cells include cells that are not yet cancerous, but may become, or are likely to become cancerous.

The terms express, expressed, or expression of a nucleic acid molecule include the biosynthesis of a gene product. The term encompasses the transcription of a gene into RNA, the translation of RNA into a protein or polypeptide, and all naturally occurring post-transcriptional and post-translational modifications thereof.

Biologic activity of CYP1B1 includes, but is not limited to, enzymatic activity and metabolic activity.

The terms measure and determine are used interchangeably, and include any suitable qualitative or quantitative determinations.

It has been observed in accordance with the invention that 17β-estradiol (E2) induces cytochrome p450 1B1 (CYP1B1) expression in precancerous head and neck epithelial cells and that E2 inhibits apoptosis of precancerous head and neck epithelial cells. It has also been observed that the knockdown of CYP1B1 in precancerous head and neck epithelial cells inhibits their motility and proliferation. Motility has implications for the progression from premalignancy to malignancy, and may play a role in invasion and metastasis as cancer develops and progresses. The invention thus derives, in part, from the characterization of underlying mechanisms in the progression of epithelial cells from a precancerous to a cancerous state, with the identification of targets for chemotherapeutic intervention that can inhibit, prevent, or otherwise slow this progression. Accordingly, the invention features various methods for screening compounds or compositions for the ability to inhibit, prevent, or slow the transition of epithelial cells from a precancerous to a cancerous state. All of the methods are preferably carried out in vitro.

In one embodiment, the methods comprise contacting premalignant or malignant cells exposed to an amount of an estrogen effective to inhibit apoptosis with a test compound, and measuring the level of apoptosis in the cells in the presence of the test compound relative to the level of apoptosis in the cells in the absence of the test compound. An increase, preferably a statistically significant increase, in apoptosis in the presence of the test compound indicates that the test compound is capable of overcoming the estrogen-induced apoptosis inhibition, and restoring, at least in part, apoptosis in the premalignant cells or in the malignant cells. A further decrease in apoptosis in the presence of the test compound indicates that the test compound is capable of further inhibiting apoptosis in premalignant cells or in malignant cells in which apoptosis has been inhibited by exposure to an estrogen.

The estrogen may be natural or synthetic, and may be an estrogen hormone, including any estrogen hormone such as estradiol (E2), estriol (E3), estrone (E1), may be any phytoestrogen, may be any mycoestrogen, may be any xenoestrogen, or any combination thereof. Phytoestrogens include but are not limited to coumestans, flavonoid phytoestrogens, ligands, and isoflavones. Xenoestrogens include any chemicals that differ from natural estrogens, yet mimic the effects of natural estrogens in the body.

In parallel, premalignant cells or malignant cells exposed to an amount of an estrogen effective to inhibit apoptosis may be contacted with an agent that is known to increase apoptosis in order to serve as a positive control or as a reference value, and other premalignant cells or malignant cells exposed to an amount of an estrogen effective to inhibit apoptosis may be contacted with an agent that is known not to increase apoptosis in order to serve as a negative control.

Apoptosis may be measured according to any technique suitable in the art, including commercially available kits and assays, fluorescence or light microscopy, flow cytometry, acridine orange/ethidium bromide staining, and other suitable techniques. Preferably, the measurements are quantitative.

In some aspects, the methods comprise the step of contacting premalignant cells or malignant cells with an amount of an estrogen effective to inhibit apoptosis in the premalignant cells or malignant cells. The test compound may be contacted with the premalignant cells or malignant cells before contacting the cells with the estrogen, substantially at the same time as contacting the cells with the estrogen, or after contacting the cells with the estrogen. The period of time before or after contacting cells with the estrogen may be any suitable period of time.

In certain aspects, the methods further comprise determining whether the test compound inhibits aspects of the estrogen apoptosis inhibition pathway, including the estrogen binding to its receptor, whether the test compound itself induces apoptosis (by any known pathway through which cells may undergo apoptosis), whether the test compound reverses aspects of the estrogen inhibition, or combinations thereof.

In another embodiment, the methods comprise contacting premalignant cells expressing CYP1B1 or malignant cells expressing CYP1B1 with a test compound, and optionally an activator of CYP1B1 biologic activity, and measuring the level of motility of the cells in the presence of the test compound relative to the level of motility of the cells in the absence of the test compound. A decrease, preferably a statistically significant decrease, in motility in the presence of the test compound indicates that the test compound is capable of inhibiting motility of the premalignant cells or of the malignant cells. An increase, preferably a statistically significant increase in motility in the presence of the test compound indicates that the test compound is capable of enhancing motility of the premalignant cells or of the malignant cells. Enhancing motility may indicate that the compound is cancer promoting. Motility can be measured according to any technique suitable in the art, including commercially available kits and assays.

In parallel, premalignant cells expressing CYP1B1 or malignant cells expressing CYP1B1 may be contacted with an agent that is known to decrease motility of the cells in order to serve as a positive control or as a reference value for inhibition of motility, and other premalignant cells expressing CYP1B1 or malignant cells expressing CYP1B1 may be contacted with an agent that is known not to decrease motility of the cells in order to serve as a negative control for inhibition of motility.

In another embodiment, the methods comprise contacting premalignant cells expressing CYP1B1 or malignant cells expressing CYP1B1 with a test compound, and optionally an activator of CYP1B1 biologic activity, and measuring the level of proliferation of the cells in the presence of the test compound relative to the level of proliferation of the cells in the absence of the test compound. A decrease, preferably a statistically significant decrease in proliferation in the presence of the test compound indicates that the test compound is capable of inhibiting proliferation of the premalignant cell. An increase, preferably a statistically significant increase in proliferation in the presence of the test compound indicates that the test compound is capable of enhancing proliferation of the premalignant cell or of the malignant cell. Enhancing proliferation may indicate that the compound is cancer promoting. Proliferation can be measured according to any technique suitable in the art, including commercially available kits and assays.

In parallel, premalignant cells expressing CYP1B1 or malignant cells expressing CYP1B1 may be contacted with an agent that is known to decrease proliferation of the cells in order to serve as a positive control or as a reference value for inhibition of proliferation, and other premalignant cells expressing CYP1B1 or malignant cells expressing CYP1B1 may be contacted with an agent that is known not to decrease proliferation of the cells in order to serve as a negative control for inhibition of proliferation.

Any known activator for any known or discovered biologic activity of CYP1B1 may optionally be used in the methods, and the methods may optionally comprise using different activators or combinations of activators in order to determine if the test compound is capable of inhibiting the motility or the proliferation of the premalignant cell or of the malignant cell that is induced by or proceeds by different pathways. The test compound may be contacted with the premalignant cells or malignant cells before contacting the cells with the activator, substantially at the same time as contacting the cells with the activator, or after contacting the cells with the activator. The period of time before or after contacting cells with the CYP1B1 activator may be any suitable period of time.

In another embodiment, the methods comprise contacting premalignant cells having the CYP1B1 gene or malignant cells having the CYP1B1 gene with a test compound and an agent capable of upregulating CYP1B1 transcription, and measuring the level of CYP1B1 mRNA in the cells in the presence of the test compound relative to the level of CYP1B1 mRNA in the cells in the absence of the test compound. A decrease, preferably a statistically significant decrease in the level of CYP1B1 mRNA in the presence of the test compound indicates that the test compound is capable of inhibiting CYP1B1 transcription, for example, the agent-induced CYP1B1 transcription, in the premalignant cells or in the malignant cells. An increase, preferably a statistically significant increase in the level of CYP1B1 mRNA in the presence of the test compound indicates that the test compound is capable of enhancing CYP1B1 transcription, for example, the agent-induced CYP1B1 transcription, in the premalignant cells or in the malignant cells. By way of example, but not of limitation, variations of the polymerase chain reaction, nucleic acid microarrays, or gene expression profiles may be used to quantifiably measure mRNA levels.

The agent capable of upregulating CYP1B1 transcription may be an estrogen. The estrogen may be natural or synthetic, and may be an estrogen hormone, a phytoestrogen, a mycoestrogen, a xenoestrogen, or any combination thereof. Thus, in some aspects, a decrease in the level of CYP1B1 mRNA in the presence of the test compound indicates that the test compound is capable of inhibiting the estrogen-induced CYP1B1 transcription in the premalignant cells or in the malignant cells.

In parallel, premalignant cells having the CYP1B1 gene or malignant cells having the CYP1B1 gene contacted with agent capable of upregulating may be contacted with an agent that is known to decrease CYP1B1 transcription in order to serve as a positive control or as a reference value for inhibiting CYP1B1 transcription, and other premalignant cells or malignant cells having the CYP1B1 gene contacted with agent capable of upregulating may be contacted with an agent that is known not to decrease CYP1B1 transcription in order to serve as a negative control for inhibiting CYP1B1 transcription. The test compound may be contacted with the premalignant cells or malignant cells before contacting the cells with the agent, substantially at the same time as contacting the cells with the agent, or after contacting the cells with the agent. The period of time before or after contacting cells with the CYP1B1 agent may be any suitable period of time.

In an alternative embodiment, the methods comprise contacting premalignant cells having the CYP1B1 gene or malignant cells having the CYP1B1 gene with a test compound and an agent capable of upregulating CYP1B1 protein expression, and measuring the level of CYP1B1 protein in the cells in the presence of the test compound relative to the level of CYP1B1 protein in the cells in the absence of the test compound. A decrease, preferably a statistically significant decrease in the level of CYP1B1 protein levels in the presence of the test compound indicates that the test compound is capable of inhibiting CYP1B1 protein expression, for example, the agent-induced CYP1B1 expression, in the premalignant cells or malignant cells. An increase, preferably a statistically significant increase in the level of CYP1B1 protein levels in the presence of the test compound indicates that the test compound is capable of enhancing CYP1B1 protein expression, for example, the agent-induced CYP1B1 protein expression, in the premalignant cells or in the malignant cells. Enhancement of CYP1B1 protein expression may indicate that the compound is cancer promoting.

The test compound may be contacted with the premalignant cells or malignant cells before contacting the cells with the agent, substantially at the same time as contacting the cells with the agent, or after contacting the cells with the agent. The period of time before or after contacting cells with the CYP1B1 agent may be any suitable period of time.

In an alternative embodiment, the methods comprise contacting premalignant cells having the CYP1B1 gene or malignant cells having the CYP1B1 gene with a test compound and an agent capable of upregulating CYP1B1 biologic activity, and measuring the level of CYP1B1 biologic activity in the cells in the presence of the test compound relative to the level of CYP1B1 biologic activity in the cells in the absence of the test compound. A decrease, preferably a statistically significant decrease in the level of CYP1B1 biologic activity levels in the presence of the test compound indicates that the test compound is capable of inhibiting CYP1B1 biologic activity, for example, the agent-induced CYP1B1 biologic activity, in the premalignant cell or in the malignant cell. An increase, preferably a statistically significant increase in the level of CYP1B1 biologic activity in the presence of the test compound indicates that the test compound is capable of enhancing CYP1B1 biologic activity, for example, the agent-induced CYP1B1 biologic activity, in the premalignant cells or in the malignant cells. Enhancement of CYP1B1 biologic activity may indicate that the compound is cancer promoting. CYP1B1 biologic activity comprises, among other things, inducing and/or enhancing motility and/or proliferation of the premalignant cells, as well as the metabolism of estrogens and the metabolism of polyaromatic hydrocarbons, including constituents of tobacco smoke.

The test compound may be contacted with the premalignant cells or malignant cells before contacting the cells with the agent, substantially at the same time as contacting the cells with the agent, or after contacting the cells with the agent. The period of time before or after contacting cells with the CYP1B1 agent may be any suitable period of time.

In any of the methods or kits described herein, premalignant cells are preferably epithelial cells of the head or neck and preferably are cells capable of progressing from the premalignant state into a malignancy of the head and neck, though it is not necessary that the premalignant cells progress into malignant cells. In preferred aspects, the malignancy of the head and neck is a squamous cell carcinoma of the head and neck, and the premalignant cells are thus capable of progressing from the premalignant state into a squamous cell carcinoma of the head and neck. In more preferred aspects, the premalignant cells are premalignant squamous epithelial cells of the lip, oral cavity, pharynx, larynx, nasal cavity, or paranasal sinuses. The cells may be primary isolates or may be established cell lines. MSK-Leuk1 cells are a non-limiting example of premalignant oral keratinocyte cells. The malignant cells are preferably malignant squamous epithelial cells of the lip, oral cavity, pharynx, larynx, nasal cavity, or paranasal sinuses. Premalignant or malignant cells may be from any animal, with mammals such as mice, rats, and rabbits being preferred, and with humans being highly preferred.

In any of the methods or kits described herein, the methods may comprise comparing the measured effect, e.g., the level of apoptosis, the level of cell motility, the level of cell proliferation, the level of CYP1B1 mRNA, the level of CYP1B1 protein, or the level of CYP1B1 biologic activity against reference values established for each of these effects. Thus, the measured value may be compared against reference values in addition to or instead of being compared to parallel cell cultures. It is thus contemplated that over time, databases of reference values may be compiled based on screened test compounds and screening experiments and conditions, and that such databases may be used in conjunction with the methods and kits. Databases may include reference values already established in the art.

Test compounds include any purified molecule, substantially purified molecule, molecules that are one or more components of a mixture of compounds, or a mixture of a compound with any other material that can be analyzed using the methods described herein (e.g., a composition). Test compounds can be organic or inorganic chemicals, or biomolecules, and all fragments, analogs, homologs, conjugates, and derivatives thereof. Biomolecules include proteins, polypeptides, nucleic acids, lipids, monosaccharides, polysaccharides, and all fragments, analogs, homologs, conjugates, and derivatives thereof. Test compounds can be of natural or synthetic origin, and can be isolated or purified from their naturally occurring sources, or can be synthesized de novo. Test compounds can be defined in terms of structure or composition, or can be undefined. Test compounds can be an isolated product of unknown structure, a mixture of several known products, or an undefined composition comprising one or more compounds. Non-limiting examples of undefined compositions include cell and tissue extracts, growth medium in which prokaryotic, eukaryotic, or archaea cells have been cultured, and fermentation broths.

The test compound can be contacted with a cell according to any means suitable in the art, and for any suitable period of time. The test compound can be assessed at multiple concentrations.

The invention also features kits for practicing the methods. In one embodiment, a kit for screening test compounds for capability of restoring apoptosis inhibited by an estrogen in a premalignant cell or in a malignant cell comprises a premalignant or malignant squamous epithelial cell of the lip, oral cavity, pharynx, larynx, nasal cavity, or paranasal sinuses, an estrogen, and instructions for using the kit in a method for screening test compounds for capability of restoring apoptosis inhibited by an estrogen in a premalignant or malignant squamous epithelial cell of the lip, oral cavity, pharynx, larynx, nasal cavity, or paranasal sinuses. The estrogen may be any natural or synthetic estrogen hormone, phytoestrogen, mycoestrogen, xenoestrogen, or any combination thereof. The estrogen may be included at a concentration effective to induce apoptosis in a premalignant or malignant squamous epithelial cell of the lip, oral cavity, pharynx, larynx, nasal cavity, or paranasal sinuses, or the kit may further comprise instructions for dosing the estrogen and for using the estrogen to inhibit apoptosis in a premalignant or malignant squamous epithelial cell of the lip, oral cavity, pharynx, larynx, nasal cavity, or paranasal sinuses.

In one embodiment, a kit for screening test compounds for capability of inhibiting motility or proliferation of a premalignant cell or of a malignant cell comprises a premalignant or malignant squamous epithelial cell of the lip, oral cavity, pharynx, larynx, nasal cavity, or paranasal sinuses, optionally an activator of CYP1B1 biologic activity, and instructions for using the kit in a method for screening test compounds for capability of inhibiting motility of a premalignant or a malignant squamous epithelial cell of the lip, oral cavity, pharynx, larynx, nasal cavity, or paranasal sinuses and/or instructions for using the kit in a method for screening test compounds for capability of inhibiting proliferation of a premalignant or a malignant squamous epithelial cell of the lip, oral cavity, pharynx, larynx, nasal cavity, or paranasal sinuses.

In one embodiment, a kit for screening test compounds for capability of inhibiting CYP1B1 transcription in a premalignant cell or a malignant cell comprises a premalignant or malignant squamous epithelial cell of the lip, oral cavity, pharynx, larynx, nasal cavity, or paranasal sinuses having the CYP1B1 gene, an agent capable of upregulating CYP1B1 transcription, and instructions for using the kit in a method for screening test compounds for capability of inhibiting CYP1B1 transcription in a premalignant or a malignant squamous epithelial cell of the lip, oral cavity, pharynx, larynx, nasal cavity, or paranasal sinuses. The agent capable of upregulating CYP1B1 transcription may be an estrogen. The estrogen may be any natural or synthetic estrogen hormone, phytoestrogen, mycoestrogen, xenoestrogen, or any combination thereof. The premalignant or malignant squamous epithelial cell of the lip, oral cavity, pharynx, larynx, nasal cavity, or paranasal sinuses having the CYP1B1 gene may be a cell line, or may be a stably or transiently transformed cell.

In one embodiment, a kit for screening test compounds for capability of inhibiting CYP1B1 protein expression in a premalignant cell or a malignant cell comprises a premalignant or malignant squamous epithelial cell of the lip, oral cavity, pharynx, larynx, nasal cavity, or paranasal sinuses having the CYP1B1 gene, an agent capable of upregulating CYP1B1 protein expression, and instructions for using the kit in a method for screening test compounds for capability of inhibiting CYP1B1 protein expression in a premalignant or a malignant squamous epithelial cell of the lip, oral cavity, pharynx, larynx, nasal cavity, or paranasal sinuses. The agent capable of upregulating CYP1B1 protein expression may be an estrogen. The estrogen may be any natural or synthetic estrogen hormone, phytoestrogen, mycoestrogen, xenoestrogen, or any combination thereof. The premalignant or malignant squamous epithelial cell of the lip, oral cavity, pharynx, larynx, nasal cavity, or paranasal sinuses having the CYP1B1 gene may be a cell line, or may be a stably or transiently transformed cell.

In one embodiment, a kit for screening test compounds for capability of inhibiting CYP1B1 biologic activity in a premalignant cell or a malignant cell comprises a premalignant or malignant squamous epithelial cell of the lip, oral cavity, pharynx, larynx, nasal cavity, or paranasal sinuses having the CYP1B1 gene, an agent capable of upregulating CYP1B1 biologic activity, and instructions for using the kit in a method for screening test compounds for capability of inhibiting CYP1B1 biologic activity in a premalignant or a malignant squamous epithelial cell of the lip, oral cavity, pharynx, larynx, nasal cavity, or paranasal sinuses. The agent capable of upregulating CYP1B1 biologic activity may be an estrogen. The estrogen may be any natural or synthetic estrogen hormone, phytoestrogen, mycoestrogen, xenoestrogen, or any combination thereof. The premalignant or malignant squamous epithelial cell of the lip, oral cavity, pharynx, larynx, nasal cavity, or paranasal sinuses having the CYP1B1 gene may be a cell line, or may be a stably or transiently transformed cell.

The following examples are provided to describe the invention in greater detail. They are intended to illustrate, not to limit, the invention.

### EXAMPLE 1

### General Experimental Procedures

Cell lines and treatments. MSK-Leuk1 cells were derived from a dysplastic leukoplakia lesion located adjacent to a SCC of the tongue. MSK-Leuk1 cells were cultured in KGM medium (Lonza, Walkersville, Md.). MSK-Leuk1 cells (passage 33) were determined to be identical to the early passage MSK-Leuk1 cells (Identity Mapping Kit, Coriell Institute for Medical Research, Camden, N.J.). All HNSCC cell lines were derived from patients with SCC of the tongue. SCC9 (male) and SCC15 (male) cells were cultured in S-MEM medium, supplemented with 2 mM L-glutamine, 100 units/ml penicillin, 100 μg/ml streptomycin and 10% FBS. UPCI:SCC56 (male), UPCI:SCC103 (female) and UPCI:SCC122 (male) cells were cultured in MEM medium, supplemented with 2 mM L-glutamine, 100 μM non-essential amino acids, 50 μg/ml gentamycin (Gibco) and 10% FBS.

For all the experiments that involved estradiol (E2) exposure, MSK-Leuk1 cells were cultured in phenol red-free and serum-free DermaLife K Medium (Lifeline Cell Technology, Walkersville, Md.). SCC cells were cultured in their respective media with no phenol red, supplemented with charcoal-stripped serum (Gibco, Carlsbad, Calif.). Cells were incubated for 48 h to remove endogenous estrogens and then plated at 70% confluence. After 24 h, the medium was replaced with either control medium containing vehicle (0.01% ethanol) or medium supplemented with 1 nM E2 (Sigma-Aldrich, St. Louis, Mo.). Cells were harvested after the appropriate treatment period and analyzed.

Generation of CYP1B1-deficient cell lines. A set of five lentivirus-encoded shRNA constructs specific for CYP1B1 (clone id TRCN0000062323-TRCN0000062327) and the empty pLKO.1 vector (control) were obtained from Open Biosystems (Huntsville, Ala.). Each of five constructs and the pLKO.1 vector were co-transfected along with the ViraPower Lentiviral Packaging Mix (Invitrogen, Carlsbad, Calif.) into 293FT producer cells, using Lipofectamine™2000 (Invitrogen, Calif.). The viral supernatants were harvested and viral titers (10⁵-10⁶ transduction units (TU)/ml) were determined using puromycin selection of normal human fibroblasts. MSK-Leuk1 cells were incubated with different dilutions of the viral supernatants and allowed to recover in complete medium. Transfection efficiency was estimated based on transfecting cells with a construct carrying green fluorescent protein and approached 100%. Stable clones were selected using puromycin (10 μg/ml, Sigma-Aldrich, St. Louis, Mo.) and analyzed for CYP1B1 levels by Western blot.

Cell motility assay. MSK-Leuk1 cells, expressing either vector or CYP1B1 shRNA, were cultured in phenol red-free and serum-free medium for 48 h and then plated at 70% confluence. After 24 h, the cells were treated with either vehicle or E2 (1 nM) in triplicate, as described above. When cells reached 100% confluence (48 h later), the surface of the cell culture dish was carefully scratched using a micropipette tip, thus making an evenly distributed gap in the cell monolayer. The medium was replaced, and five representative images of each gap were acquired at 0 h using a Nikon TE-2000U wide field inverted microscope (Optical Apparatus Co., Ardmore, Pa.) equipped with a Roper Scientific Cool Snap HQ camera. Another set of 5-10 representative images per gap was obtained following a 16-h incubation. The area devoid of cells was measured on every image using MetaMorph 7.0 (Molecular Devices, Inc., Sunnyvale, Calif.). The gap closure percentages were calculated as (area at 0 h−area at 16 h)/(area at 0 h).

In addition, a time-lapse movie capturing the process of gap closure in vector-expressing MSK-Leuk1 cells was obtained. The medium was replaced with fresh medium containing 25 mM HEPES buffer, and cells were allowed to incubate for 1 h at 37° C. A preset location was photographed every 10 min for a period of 16 h using the same microscope and camera set-up as above. The percentage of proliferating cells (those rounded up for cell division) was counted in this representative area.

Apoptosis assay. Apoptosis was assessed using the Guava Nexin® (Millipore Corp., Billerica, Mass.). Fifty thousand cells were plated per well in 6-well plates. After the appropriate treatment, floating cells were collected, combined with attached cells following trypsinization, and resuspended in DermaLife® K Medium (Lifeline Cell Technology, Walkersville, Md.) supplemented with 5% FBS. The cell suspension (100 μl) was incubated with 100 μl of Guava Nexin® Reagent for 20 min, according to the manufacturer's instructions. Two thousand cells were analyzed from each sample using the Guava EasyCyte™ system, and the resulting data were expressed as a percentage of apoptotic cells (annexin V positive cells/total number of cells counted).

Cell proliferation. Fifty thousand cells/well were plated in 6-well plates. After the appropriate treatment, the DNA content of the cells, an indirect measure of proliferation, was determined using a Fluorescent DNA Quantitation kit (Bio-Rad Laboratories, Hercules, Calif.). In brief, cells were harvested, sonicated in 0.1× TEN assay buffer (Bio-Rad Laboratories) for 5 s, and incubated with a Hoechst dye mixture (BioRad Laboratories) for 1 h. Total DNA was measured using Fluoroscan Ascent FL (Thermo Fisher Scientific, Waltham, Mass.) at an excitation wavelength of 360 nm and an emission wavelength of 460 nm.

Tissue microarrays. Tissue microarrays (TMAs) contained duplicate tissue cores of surgical head and neck specimens from 128 patients, including 116 samples of HNSCC, 20 samples of dysplasia and 37 samples of normal epithelium from different sites within the head and neck. Mean age at diagnosis was 64 years (range 30-90 years). Sixty-nine percent of the patients were males and 24% were females, with gender unknown for the remaining 7% of patients. The characteristics of this population are summarized in Table 1, below.

Immunohistochemical staining and quantification. Immunohistochemical analyses were performed on histological sections of formalin-fixed, paraffin-embedded human head and neck TMAs and cytospins of cultured human head and neck cells. Sections were stained with antibodies against human CYP1B1 (raised in rabbit, Alpha Diagnostics International Inc., San Antonio, Tex.), estrogen receptor (ER)α (raised in mouse, Lab Vision Products, Fremont, Calif.), ERβ (raised in mouse, Serotec, Raleigh, N.C.) and E2 (raised in rabbit, BioGenex, San Ramon, Calif.) using standard immunohistochemical procedures (QualTek Molecular Laboratories, Newtown, Pa.). Sodium citrate (pH 6) was used for antigen retrieval. Human breast carcinoma was used as a positive control for each antibody. TMA sections were scanned and images were captured using an Automated Cellular Imaging System (ACIS, ChromaVision, San Juan Capistrano, Calif.). Pathologically confirmed regions of HNSCC, dysplasia and normal epithelium were scanned using a 40× objective. Following normalization to a threshold (background staining), the staining intensity of each selected area was quantified and expressed in arbitrary units.

Protein extraction and Western blotting. Cells were solubilized in RIPA buffer containing 150 mM NaCl, 1% Na Deoxycholate, 1% Triton® X-100, 0.1% SDS, 10 mM Tris-Base, 50 mM NaF, 0.1 mM Na3VO4, supplemented with protease inhibitor cocktail (Roche, Indianapolis, Ind.). One hundred micrograms of total protein was separated on a 10% sodium dodecyl sulfate polyacrylamide gel (Bio-Rad, Hercules, Calif.) and electroblotted onto a polyvinylidene fluoride membrane. Membranes were blocked for 1 h at room temperature in Tris-buffered saline with Tween®-20 (TBST) (50 mM Tris-HCl, pH 7.5, 150 mM NaCl, 0.1% Tween®-20) containing 5% nonfat milk and incubated overnight at 4° C. with primary antibodies against either CYP1B1 (Imgenex Corp., San Diego, Calif., Catalog No: IMG-5988A), ERα (Santa-Cruz Biotechnology Inc., Santa Cruz, Calif., H-184:sc-7207), ERβ (Millipore, Billerica, Mass., Catalog No: 05-824) or HPRT (Abcam, Inc., Cambridge, Mass., Catalog No: ab10479). After washing 3 times with TBST, the membranes were incubated with horseradish peroxidase (HRP)-conjugated goat anti-rabbit IgG secondary antibody (Bio-Rad) (1 h at room temperature), rinsed with TBST and visualized using ECL Western Blotting Detection Reagents (GE Healthcare, Piscataway, N.J.).

Quantitative real-time reverse transcription PCR. RNA was extracted from pelleted cultured cells using the RNeasy Mini kit (Qiagen, Valencia, Calif.) and its quality (18S and 28S RNA) was evaluated by gel electrophoresis. cDNA was synthesized from 1 μg of total RNA, using the High Capacity cDNA Archive Kit (Applied Biosystems, Inc., Foster City, Calif.). Three μl of the resulting cDNA were mixed with 12.5 μl of 2× TaqMan® Universal PCR master mix and 1.25 μL of 20× primer mix (Applied Biosystems) in a final reaction volume of 25 μL, according to the manufacturer's instructions. Reactions were performed in triplicate in an Applied Biosystems 7900HT Fast Real-Time PCR System using universal conditions. Transcript levels were quantified and expressed relative to those of the human transferrin receptor (TFRC) as 2^{-ΔCt}.

Statistical analyses. For the gene expression, cell proliferation and apoptosis analyses, Student's t-test (Excel) was used to analyze differences between the groups. For the cell migration assay, statistical analyses were performed using the two-sided Mann-Whitney test (Instat Statistical Software, GraphPad Software, San Diego, Calif.).

TMAs were analyzed by comparing the staining intensity of each antibody in HNSCCs, dysplasias and normal head and neck epithelium using a pairwise approach. When appropriate, the staining intensities of each antibody were compared within each tissue type by gender. All TMA statistical analyses were done using the two-sided Mann-Whitney test. The Benjamini-Hochberg false discovery rate approach was used to account for multiple testing. All P values listed for TMA analyses were corrected P values based on this approach. The R statistical language and environment was used for these analyses.

### EXAMPLE 2

### Experimental Results

A. Estrogen metabolism genes and ERβ are expressed in cells derived from premalignant and malignant head and neck lesions.

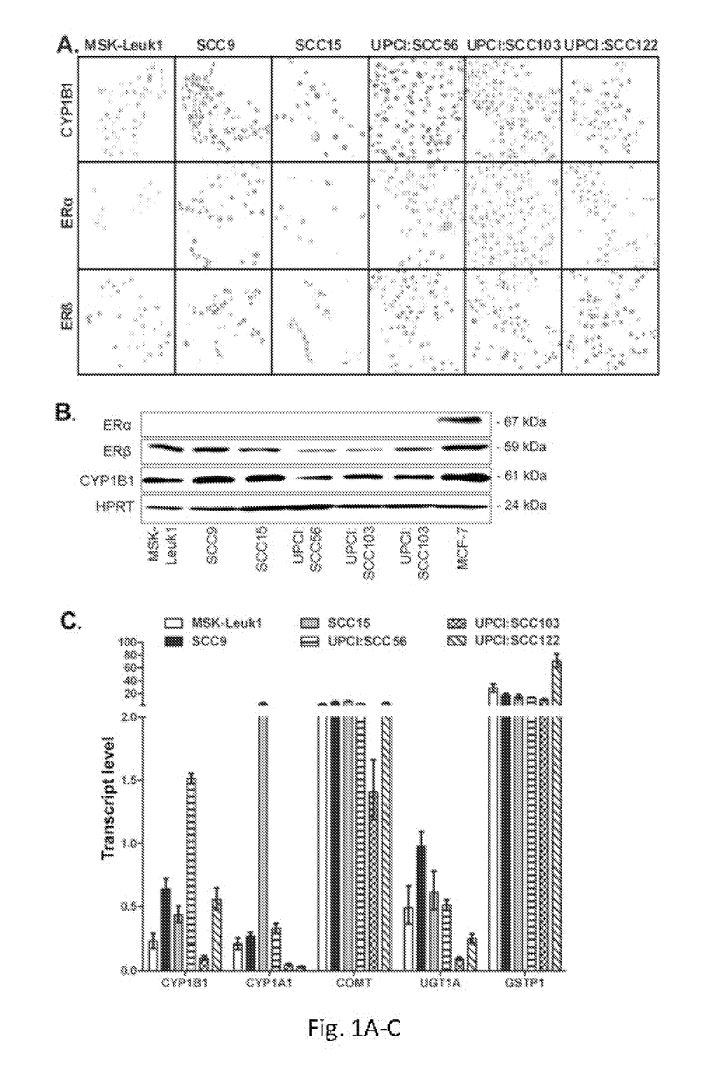

Immunohistochemical staining of sections from formalin-fixed, paraffin embedded pellets of MSK-Leuk1 cells and five HNSCC cell lines was performed using antibodies specific for ERα, ERβ and CYP1B1. ERβ and CYP1B1 were detected in MSK-Leuk1 cells and all HNSCC cell lines at comparable levels, with staining for both proteins localized to the nucleus. ERα was not detected in any of the cell lines evaluated (FIG. 1A). Consistent with immunohistochemical staining data, ERβ and CYP1B1 were detected by Western blot in all head and neck lines. While ERα was detected in MCF-7 cells (positive control), it was not detectable in any of the head and neck cell lines (FIG. 1B).

The finding that ERβ and CYP1B1 are present in cultured head and neck cells was extended by examining the expression profile of CYP19 (aromatase), which encodes the rate-limiting enzyme in estrogen synthesis, and several estrogen metabolism genes in both MSK-Leuk1 cells and five HNSCC cell lines. Transcripts for CYP1B1, CYP1A1, COMT, UGT1A, and GSTP1 were detected in all cell lines (FIG. 1B), while transcripts for CYP3A4 and SULT1A1 were near the limits of detection (not shown). The most abundant transcripts in all cell lines were those encoding the conjugation genes COMT and GSTP1. The level of CYP19 transcripts was below the limit of detection in all cell lines evaluated (data not shown).

B. E2 induces the expression of CYP1B1 in cells derived from premalignant but not malignant head and neck lesions.

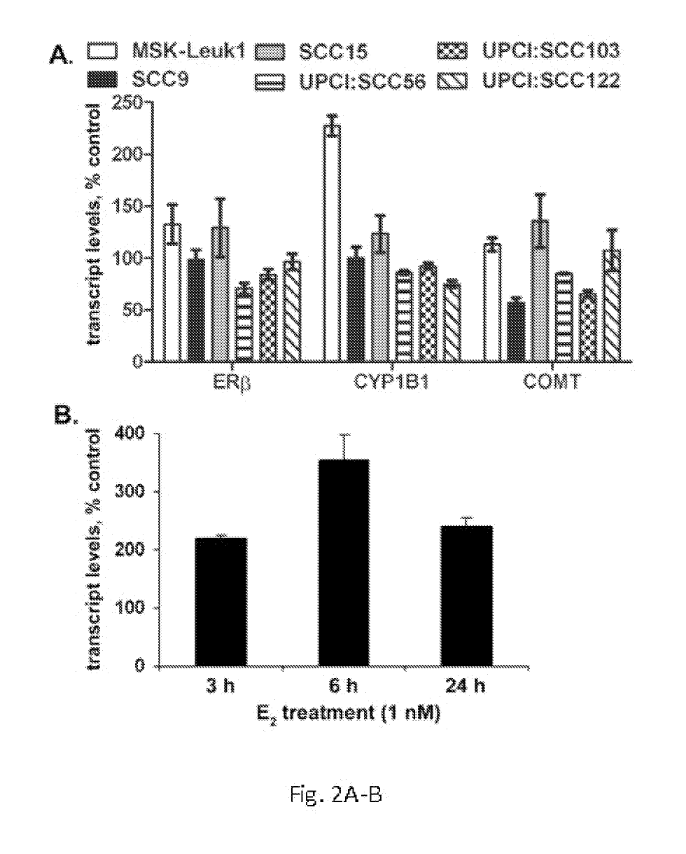

The effect of E2 exposure on transcript levels of ERβ, CYP1B1 and COMT, that encodes the major estrogen and xenobiotic conjugation enzyme, was examined in cultured MSK-Leuk1 and HNSCC cells. In MSK-Leuk1 cells, E2 treatment (24 h) induced the levels of CYP1B1 transcripts 2.3-fold over that of vehicle-treated controls (P=0.0004) (FIG. 2A). The induction of CYP1B1 by E2 appeared to be time dependent in MSK-Leuk1 cells, and peaked after 6 h of exposure (FIG. 2B). In contrast, the levels of COMT, ERβ, as well as the reference gene TFRC, remained unaltered in MSK-Leuk1 cells post-treatment. Treatment of HNSCC cells with E2 did not alter the level of any of the transcripts of interest (ERβ, CYP1B1 and COMT).

C. CYP1B1 deficiency decreases the motility of MSK-Leuk1 cells.

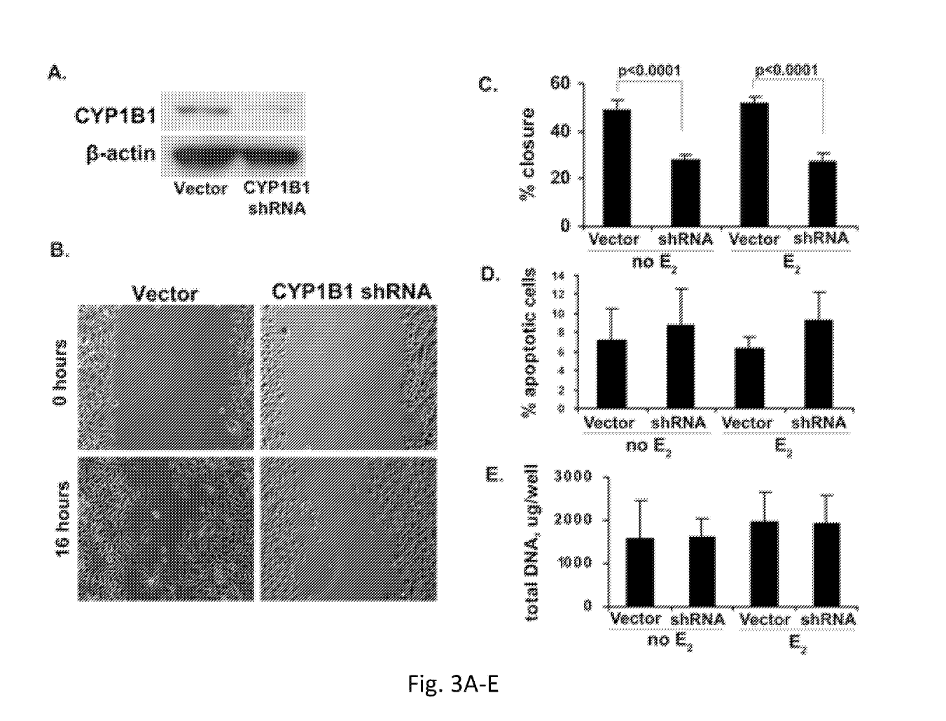

To investigate the contribution of CYP1B1 to cancer progression, MSK-Leuk1 cells deficient in CYP1B1 were constructed using a lentivirus system to express shRNA specific to CYP1B1 mRNA. Western blot analyses indicated that CYP1B1 levels were decreased in cells expressing CYP1B1 shRNA, relative to control cells that expressed the vector (FIG. 3A).

The motility of CYP1B1-deficient MSK-Leuk1 cells was compared to that of cells expressing control vector (treated with either vehicle or E2). The rate of motility of CYP1B1-deficient cells measured as the ability of the cells to repopulate a scratched area of a previously confluent monolayer, was 54-57% lower than that of control cells expressing the basic vector (P<0.0001; FIG. 3, B and C). Motility was not affected by E2 treatment. Rates of proliferation and apoptosis were comparable in CYP1B1 shRNA- and vector-expressing cells during the time period when cell migration was analyzed (16 h; see FIG. 3, D and E).

To confirm that the observed gap closure was due to the migration and not proliferation of the cells, the motility of vector-expressing MSK-Leuk1 cells was observed in real time over a 16-h period. The cells were motile, with approximately 20% dividing during the observation period. No difference in proliferative rate was observed among the cells infiltrating the gap, as compared to the cell monolayer outside of the gap (data not shown).

D. The effects of E2 exposure on the proliferation and apoptosis of cultured MSK-Leuk1 cells with or without CYP1B1 knockdown.

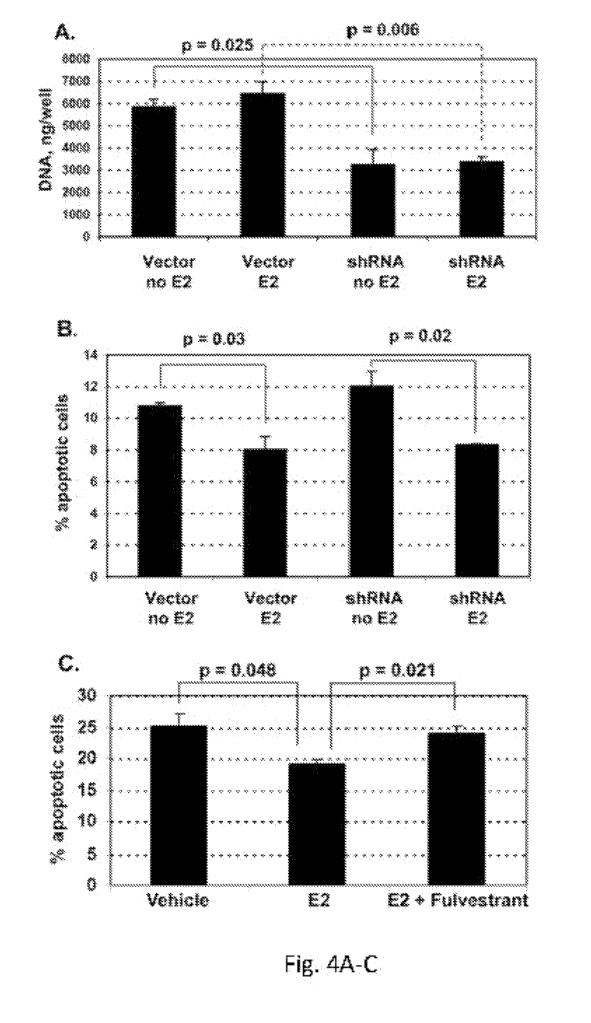

To explore the role of E2 in head and neck carcinogenesis, MSK-Leuk1 cells expressing either vector or CYP1B1 shRNA were incubated in the presence or absence of E2 for 72 h. The proliferation of cells expressing CYP1B1 shRNA was decreased as compared to that of vector-expressing cells irrespective of E2 exposure (44.6% for vehicle-treated cells (P=0.025) and 47.6% for E2-treated cells (P=0.006), FIG. 4A). E2 exposure induced cell proliferation in vector-expressing cells by 10%; this increase, however, was not statistically significant (FIG. 4A). CYP1B1 depletion did not affect apoptosis (FIG. 4B). Exposure to E2, however, decreased apoptosis in both vector-expressing (by 25.5%, P=0.030) and CYP1B1 shRNA-expressing (by 30.1%, P=0.015) cells (FIG. 4B). This E2-mediated decrease in apoptosis was restored by the addition of the pure antiestrogen fulvestrant (FIG. 4C).

E. E2, CYP1B1 and ERβ are detected in normal, dysplastic and SCC tissues of the head and neck, with the levels of CYP1B1 and ERβ elevated significantly in HNSCCs.

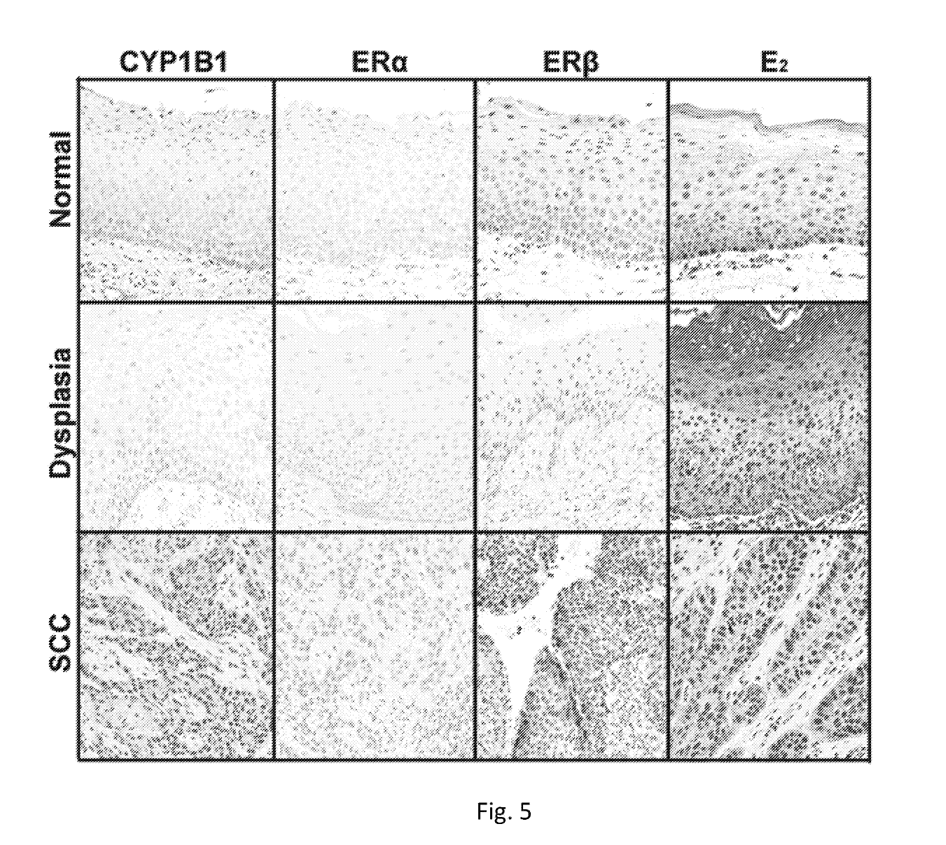

Representation of the estrogen pathway in human head and neck tissue was examined using TMAs of head and neck surgical specimens. TMAs were stained with antibodies against ERα, ERβ, CYP1B1 and E2. The majority of samples stained positive for ERβ (91.9%), CYP1B1 (99.4%) and E2 (88.4%), irrespective of gender. Staining of ERβ and CYP1B1 was localized to the nucleus, while staining of E2 was observed in both the nucleus and cytoplasm (FIG. 5). Staining of ERα was detected in only a few cases (1.7%).

The staining intensity of ERβ, CYP1B1 and E2 was quantified in pathologically confirmed areas of cancer, dysplasia and normal epithelium using the ACIS (ChromaVision). Staining intensity of CYP1B1 and ERβ were both higher in HNSCCs as compared to normal epithelium (P=0.024 and 0.008, respectively, FIG. 5, Table 1). No difference in the intensity of E2 staining was observed between HNSCCs, dysplasias or normal epithelium (FIG. 5, Table 1). In addition, no difference between males and females was observed in the staining intensity of any of the antibodies when either normal epithelium or HNSCCs were analyzed. Because HPV infection is associated with a better prognosis in HNSCC patients, the analyses were next restricted to sites of the head and neck not routinely associated with HPV infection. Similar to the results obtained when evaluating all specimens, the intensity of ERβ staining in the potentially non HPV-associated tumors was elevated as compared to normal epithelium (P=0.007). CYP1B1 staining intensity was also elevated in HNSCCs relative to normal epithelium and approached statistical significance (P=0.07). No difference in the intensity of E2 staining was observed between HNSCC, dysplasia or normal epithelium for the potentially non HPV-associated cases.

TABLE 1
Comparison of the immunohistochemical staining intensities of CYP1B1,
ERβ and E2 in human head and neck tissues.

| | Median Intensity (± Standard Error) | | | |
|---|---|---|---|---|
| Antibody | Normal | Dysplasia | Cancer | P* |
| CYP1B1 | 108.5 (±3.5) | 109.8 (±4.4) | 123.8 (±1.9) | 0.024 |
| ERβ | 104.8 (±3.8) | 113.9 (±4.6) | 131.7 (±2.4) | 0.008 |
| E2 | 100.4 (±5.25) | 99.3 (±5.9) | 95.8 (±2.3) | 0.168 |

*P values are for normal versus cancer comparisons and were corrected for multiple comparisons.

### EXAMPLE 3

### Summary

The results demonstrate that a panel of estrogen metabolism genes is expressed in cultured human head and neck cells. Without intending to be limited to any particular theory or mechanism of action, it is believed that detection of transcripts for these genes in both premalignant lesions and HNSCCs suggests that these enzymes contribute to cellular metabolism throughout tumorigenesis. It is believed that to date, the contribution of the estrogen pathway to the premalignant stage of head and neck tumorigenesis has not been evaluated.

The results show that CYP1B1 is upregulated in MSK-Leuk1 but not in HNSCC cells following E2 exposure. The mechanistic basis for this differential upregulation of CYP1B1 remains unclear. However, it has been shown for lung cancer that the timing of hormone exposure relative to a diagnosis of lung cancer may make a difference with respect to whether the hormonal effect is protective or adverse (Siegfried J M (2010) Cancer Prev. Res. 3:692-5). CYP1B1 metabolizes hormones, including E2, and xenobiotics, including tobacco-associated carcinogens, to species that can cause DNA damage. Without intending to be limited to any particular theory or mechanism of action, it is believed that enhanced expression of CYP1B1, in the absence of an elevation in the expression of the conjugation gene COMT, could potentially promote the accumulation of mutagenic DNA damage and contribute to the formation of HNSCCs.

To assess the functional role of CYP1B1 in premalignant head and neck cells, the migratory potential of MSK-Leuk1 cells deficient in CYP1B1 was compared to that of the same cell line carrying control vector. The observed decrease in cell migration was not attributed to decreased proliferation (FIG. 3). However, when a longer exposure time was investigated (72 hours, FIG. 4), cell proliferation was decreased in CYP1B1-deficient cells, as compared to vector-expressing cells. Without intending to be limited to any particular theory or mechanism of action, it is believed that the ability of CYP1B1, independent of E2, to promote the migration and proliferation of oral premalignant cells may play a role in the clonal spread of leukoplakic lesions within the oral mucosa and facilitate cancer progression within the head and neck.

Exposure to E2 failed to alter the rate of cell proliferation in MSK-Leuk1 cells, irrespective of CYP1B1 levels. In contrast, E2 inhibited apoptosis in both control and CYP1B1-deficient MSK-Leuk1 cells. Without intending to be limited to any particular theory or mechanism of action, it is believed that the observed ability of E2 to decrease apoptosis in premalignant cultured cells suggests that estrogens may be involved in the progression of premalignant lesions to HNSCCs. The ability of the pure antiestrogen fulvestrant to antagonize E2-mediated inhibition of apoptosis, suggests that this effect is ER-mediated and that antiestrogens may be beneficial as chemopreventive agents for HNSCC.

Using TMAs of surgical specimens from 128 patients, it was observed that CYP1B1 protein is present at detectable levels in normal, dysplastic and tumor tissues of the head and neck. Previously, CYP1B1 mRNA and/or protein have been detected in HNSCC cell lines (Chi A C, et al. (2009) Oral Oncol. 45:980-5; Walle T et al. (2007) J. Pharm. Pharmacol. 59:857-62) and MSK-Leuk1 cells (Hughes D et al. (2008) Cancer Prev. Res. 1:485-93; Boyle J O et al. (2010) Cancer Prev. Res. 3:266-78); it is believed, however, that human head and neck tissues have not previously been analyzed for CYP1B1 protein. The results show that CYP1B1 is overexpressed in HNSCCs as compared to the normal epithelium of the head and neck. It is believed that CYP1B1 may be a marker of head and neck tumorigenesis, as evidenced by the enhanced expression of CYP1B1 in HNSCCs.

MSK-Leuk-1 cells, as well as cultured HNSCC cells, stained positive for ERβ expression. Consistent with these data, ERβ was detected in human HNSCCs and dysplastic tissues as well as in the normal epithelium. The detection of ERβ in both dysplastic and HNSCC cells suggests the potential contribution of estrogen signaling to the development of HNSCCs at both the premalignant and malignant stages. The absence of a gender difference in the intensity of immunohistochemical staining for CYP1B1, ERβ or E2 suggests that the estrogen pathway may contribute to head and neck carcinogenesis in both males and females.

The invention is not limited to the embodiments described and exemplified above, but is capable of variation and modification within the scope of the appended claims.

1-8. (canceled)

9. A method for screening compounds for the ability to modulate CYP1B1-mediated motility or proliferation in a premalignant or malignant cell expressing CYP1B1, comprising: contacting premalignant cells expressing CYP1B1 or malignant cells expressing CYP1B1 with a test compound and measuring the level of motility or proliferation of the premalignant or malignant cells in the presence of the test compound relative to the level of motility or proliferation of the cells in the absence of the test compound, wherein a decrease in motility or proliferation in the presence of the test compound indicates that the test compound is capable of inhibiting motility or is capable of inhibiting proliferation of the premalignant cells or of the malignant cells, and wherein an increase in motility or proliferation of the cells in the presence of the test compound indicates that the test compound is capable of enhancing motility or is capable of enhancing proliferation of the premalignant cells or of the malignant cells.

10. (canceled)

11. The method of claim 9, wherein the premalignant cells are capable of progressing to a malignancy of the head and neck.

12. The method of claim 11, wherein the malignancy of the head and neck is a squamous cell carcinoma of the head and neck.

13. The method of claim 9, wherein the premalignant cells are premalignant squamous epithelial cells of the lip, oral cavity, pharynx, larynx, nasal cavity, or paranasal sinuses.

14. The method of claim 9, wherein the malignant cells are squamous cell carcinoma of the head and neck cells.

15-46. (canceled)

47. A method for screening compounds for the ability to modulate CYP1B1 biologic activity in a premalignant cell expressing the CYP1B1 gene or in a malignant cell expressing the CYP1B1 gene, comprising: contacting premalignant cells having the CYP1B1 gene or malignant cells having the CYP1B1 gene with a test compound, and measuring the level of CYP1B1 biologic activity in the premalignant or malignant cells in the presence of the test compound relative to the level of CYP1B1 biologic activity in the premalignant or malignant cells in the absence of the test compound, wherein a decrease in the level of CYP1B1 biologic activity in the presence of the test compound indicates that the test compound is capable of inhibiting CYP1B1 biologic activity in the premalignant cells or in the malignant cells, and wherein an increase in the level of CYP1B1 biologic activity in the presence of the test compound indicates that the test compound is capable of enhancing CYP1B1 biologic activity in the premalignant cells or in the malignant cells.

48. (canceled)

49. The method of claim 47, wherein the premalignant cells are capable of progressing to a malignancy of the head and neck.

50. The method of claim 49, wherein the malignancy of the head and neck is a squamous cell carcinoma of the head and neck.

51. The method of claim 47, wherein the premalignant cells are premalignant squamous epithelial cells of the lip, oral cavity, pharynx, larynx, nasal cavity, or paranasal sinuses.

52. The method of claim 47, wherein the malignant cells are squamous cell carcinoma of the head and neck cells.

53. (canceled)

54. The method of claim 47, wherein a decrease in the level of CYP1B1 biologic activity in the presence of the test compound indicates that the test compound is capable of inhibiting the agent-induced CYP1B1 biologic activity in the premalignant cells or in the malignant cells.

55. The method of claim 47, wherein the agent capable of upregulating CYP1B1 biologic activity is an estrogen.

56. The method of claim 55, wherein the estrogen is an estrogen hormone, phytoestrogen, mycoestrogen, xenoestrogen, or combination thereof.

57. The method of claim 55, wherein a decrease in the level of CYP1B1 biologic activity in the presence of the test compound indicates that the test compound is capable of inhibiting the estrogen-induced CYP1B1 biologic activity in the premalignant cells or in the malignant cells.

58. The method of claim 57, wherein the estrogen is an estrogen hormone, phytoestrogen, mycoestrogen, xenoestrogen, or combination thereof.

59-67. (canceled)

68. The method of claim 47, wherein the CYP1B1 biologic activity comprises promoting the motility of premalignant or malignant epithelial cells of the head and neck.

69. The method of claim 47, wherein the CYP1B1 biologic activity comprises promoting the proliferation of premalignant or malignant epithelial cells of the head and neck.

70. A kit, comprising a premalignant or malignant squamous epithelial cell of the lip, oral cavity, pharynx, larynx, nasal cavity, or paranasal sinuses, said cell expressing the CYP1B1 gene, and instructions for using the kit in the method of claim 9.

71. A kit, comprising a premalignant or malignant squamous epithelial cell of the lip, oral cavity, pharynx, larynx, nasal cavity, or paranasal sinuses, said cell expressing the CYP1B1 gene, and instructions for using the kit in the method of claim 47.

72. The kit of claim 71, further comprising an estrogen selected from the group consisting of estrone, estradiol, estriol, a phytoestrogen, a mycoestrogen, and a xenoestrogen.
